(19) United States
(12) Patent Application Publication
(10) Pub. No.: US 2023/0414560 A1
(43) Pub. Date: Dec. 28, 2023

(54) COMPOSITIONS AND METHODS FOR TREATING ECZEMA

(71) Applicant: General Nutraceutical Technology LLC, Elmsford, NY (US)

(72) Inventors: Xiu-Min Li, Cortlandt Manor, NY (US); Nan Yang, Mamaroneck, NY (US); Zhenzhen Wang, Zhenzhou City (CN)

(21) Appl. No.: 18/323,032

(22) Filed: May 24, 2023

Related U.S. Application Data
(60) Provisional application No. 63/345,702, filed on May 25, 2022.

(51) Int. Cl. A61K 31/353; A61P 17/00; A61K 31/231; A61K 31/232; A61K 31/015; A61K 31/575; A61K 31/122; A61K 31/19; A61K 31/215; A61K 31/365; A61K 31/366; A61K 31/12; A61K 31/7048; A61K 31/404; A61K 9/06; A61K 36/076; A61K 36/65; A61K 36/21; A61K 36/355; A61K 36/30; A61K 36/634; A61K 36/48; A61K 36/484; A61K 36/185; A61K 36/232; A61K 36/489

(52) U.S. Cl. CPC A61K 31/353; A61P 17/00; A61K 31/231; A61K 31/232; A61K 31/015; A61K 31/575; A61K 31/122; A61K 31/19; A61K 31/215; A61K 31/365; A61K 31/366; A61K 31/12; A61K 31/7048; A61K 31/404; A61K 9/06; A61K 36/076; A61K 36/65; A61K 36/21; A61K 36/355; A61K 36/30; A61K 36/634; A61K 36/48; A61K 36/484; A61K 36/185; A61K 36/232; A61K 36/489

(57) ABSTRACT

A pharmaceutical composition for the treatment of eczema is disclosed. Also disclosed are methods of treating eczema in a subject by administering a pharmaceutical composition.

### CROSS REFERENCE TO RELATED APPLICATION

This application claims the benefit of priority from U.S. Provisional Application No. 63/345,702, filed on May 25, 2022, the contents of which are incorporated herein by reference.

### FIELD OF THE DISCLOSURE

The present disclosure relates to compositions and methods for treating eczema.

### INCORPORATION BY REFERENCE OF SEQUENCE LISTING

The Sequence Listing in an XML format, named as 40921_SequenceListing.xml of 14 KB, created on May 24, 2023, and submitted to Patent Center, is incorporated herein by reference.

### BACKGROUND

Eczema is a chronic inflammatory skin disorder accompanied by redness, edema, dryness, itching, and lichenification of the skin. Up to 20% of children and 1-3% of adults in the United States are suffering from eczema and co-morbidities including allergic rhinitis, food allergy, and asthma (Novak, 2009, Allergy, 64(2), 265-275), severely impacting their quality of life and social activities. The annual cost of eczema in the United States has been estimated over $5.297 billion USD in 2015 (Drucker et al., 2017, J Invest Dermatol, 137(1), 26-30). Various immune cells and cytokines participate in different levels of eczema, such as Th2 cytokines (IL-4, IL-5, and IL-13), Th17 (IL-17A, IL-17F, and IL-22), and Th9 cells (Klonowska et al., 2018, Int J Mol Sci, 19(10). Thus, treatment specifically focusing on the single cytokine fail to improve the symptoms of severe eczema patients. To date, topical corticosteroids (steroids) are still first-line treatment in the clinic through oral steroids accompanied by multiple side effects such as weight gain, edema, nausea, and sleep disturbance (Barnes, 2006, Eur J Pharmacol, 533(1-3), 2-14). Further, systemic treatments are required for steroid-refractory eczema patients (Wang, Z. et al., 2021, Ann Allergy Asthma Immunol, 126, 639-654). Thus, the development of new treatments to effectively improve the skin symptoms and balance the immune system is in urgent need.

Traditional Chinese Medicine (TCM) has been used in skin diseases for centuries. The effective TCM formula, such as Xiao-Feng-San (XFS) (Chen et al., 2016, ComplementAltern Med, 16, 173), Pei-Tu-Qing-Xin-Tang (PTQXT) (Liu, J. et al., 2015, PloS one, 7(9), e43918-e43918), Qin-Zhu-Liang-Xue decoction (Ma et al., 2020, Ann Palliat Med, 9(3), 870-882), and Run-Zao-Zhi-Yang capsules (RZZYC) (Huang et al., 2019, Journal of Dermatological Treatment, 30(7), 677-684), have achieved significant success in improving quality of life, improving severity scores, reducing pruritus, and decreasing corticosteroid use by regulating multiple pathways (Chen et al., 2015, J Ethnopharmacol, 159, 189-196). A multiple TCM therapy approach, Shi-Zhen-Tea (SZT), including oral herbal internal supplements (Shi Zhen Tea I and Shi Zhen Tea Ia), cream (cream I, and cream II), and bath (bath I) (Wang et al, 2021, Ann Allergy Asthma Immunol. 126, 639-654; Wang et al, 2021, Evid Based Complement Alternat Med, 13, 8406127) has been successfully used as complementary treatment in the US for moderate-to-severe eczema (Srivastava et al., 2020; The Journal ofAllergy and Clinical Immunology, 145(2), AB198; Thanik et al., 2018, Ann Allergy Asthma Immunol, 121(1), 135-136). Shi Zhen Tea I are used for acute eczema patients with redness, itching, woozv, and rash. However, Shi Zhen Tea Ia (Wang et al, 2021, Ann Allergy Asthma Immunol. 126, 639-654; Wang et al, 2021, Evid Based Complement Alternat Med, 13, 8406127) are usually used for chronic eczema.

### BRIEF SUMMARY OF THE DISCLOSURE

One aspect of the present disclosure is directed to a pharmaceutical composition for treating eczema, comprising a therapeutically effective amount of at least one purified compound selected from Table 1, and a pharmaceutical carrier.

In some embodiments, the at least one purified compound comprises a combination of purified compounds selected from Table 1.

In some embodiments, the combination of purified compounds selected from Table 1 comprises at least two purified compounds selected from Table 1. In some embodiments, the combination of purified compounds selected from Table 1 comprises at least three purified compounds selected from Table 1. In some embodiments, the combination of purified compounds selected from Table 1 comprises at least four purified compounds selected from Table 1.

In some embodiments, the at least one purified compound is selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

In some embodiments, the at least one purified compound is selected from quercetin, luteolin or kaempferol. In some embodiments, the at least one purified compound is luteolin.

In some embodiments, the at least one purified compound comprises a combination of purified compounds selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

In some embodiments, the at least one purified compound comprises a combination of purified compounds selected from quercetin, luteolin or kaempferol. In some embodiments, the combination of purified compounds includes luteolin.

In some embodiments, the pharmaceutical composition is formulated for oral administration. In some embodiments, the pharmaceutical composition is formulated as a cream.

In some embodiments, the pharmaceutical composition is in the form of a dry powder.

Another aspect of the disclosure is directed to a pharmaceutical composition for treating eczema comprising (i) at least one purified compound selected from Table 1 formulated for oral administration, (ii) at least one purified compound selected from Table 1 formulated as a cream, and/or (iii) at least one purified compound selected from Table 1 formulated as a powder for soak.

In some embodiments, the at least one purified compound for each of (i), (ii) and (iii) is independently selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

In some embodiments, the at least one purified compound for each of (i), (ii) and (iii) is independently selected from quercetin, luteolin or kaempferol. In some embodiments, the at least one purified compound for each of (i), (ii) and (iii) is luteolin.

Another aspect of the disclosure is directed to a method of treating eczema in a subject comprising administering to the subject a pharmaceutical composition which comprises a therapeutically effective amount of at least one purified compound selected from Table 1 and a pharmaceutical carrier.

In some embodiments, the pharmaceutical composition is administered orally. In some embodiments, the pharmaceutical composition is administered by applying on a skin in the form of a cream. In some embodiments, the pharmaceutical composition is in the form of a dry powder and administered by applying on a skin in a soak.

Another aspect of the disclosure is directed to a method of treating eczema in a subject comprising administering to the subject (i) at least one purified compound selected from Table 1 via oral administration, (ii) at least one purified compound selected from Table 1 via topical administration as a cream, and/or (iii) at least one purified compound selected from Table 1 as a powder for soak.

In some embodiments, the at least one purified compound comprises a combination of purified compounds selected from Table 1.

In some embodiments, the at least one purified compound for each of (i), (ii) and (iii) is independently selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

In some embodiments, the at least one purified compound for each of (i), (ii) and (iii) is independently selected from quercetin, luteolin or kaempferol.

In some embodiments, the at least one purified compound for each of (i), (ii) and (iii) is luteolin.

### BRIEF DESCRIPTION OF THE DRAWINGS

The patent or application file contains drawings executed in color. Copies of this patent or patent application publication with color drawing(s) will be provided by the Office upon request and payment of the necessary fee.

FIGS. 1A-1D. Sharpin^{cpdm/cpdm}m genotyping and dermatitis in Sharpin^{cpdm/cpdm} am mouse after SZT-I treatment. (A) Representative gel for genotyping of heterozygote, sharpin gene knockout, and wild type mice; (B) Treatment protocol; Representative progression of dermatitis in Sharpin^{cpdm/cpdm} mice beginning at 6 weeks of sham group (C), and treatment group (D).

FIGS. 2A-2D. The efficacy of SZT-I treatment on dermatitis in Sharpin^{cpdm/cpdm} mice. (A). Atopic Dermatitis Scoring criteria; (B). Extent of scratching; (C). Percentage of body surface lesions area; (D) Intensity of skin lesions score based on SCORAD-M guide for treated and sham mice. Sham vs Treated, *p<0.05, **p<0.01, ***p<0.001.

FIGS. 3A-3E. H+E Histology samples and corresponding eosinophils counts. Histology samples of skin (A) and liver (B) from the sham mice; Histology samples of skin (C) and liver (D) from the treated mice. (E) Eosinophil numbers count in sham and treated mice. Significance is indicated as **p<0.01; *** p<0.001 compared to sham samples.

FIGS. 4A-4J. In vitro anti-TNF-α effects, anti-IgE, cytotoxicity and anti-inflammatory effects of SZT-I. (A) Anti-TNF-α effects of SZT-I on Raw 264.7 cells; (B) The IC-50 value calculated based on X=log[x] transformed data from D; (C) SZT-I Raw 264.7 cytotoxicity; (D) Anti-IgE effects of SZT-I on U266 cells; (E) The IC-50 value calculated based on X=log[x]transformed data from A; (F) SZT-I U266 cytotoxicity; Significance is indicated as *p<0.05, **p<0.01, ***p<0.001 compared to media control; (G) Anti-inflammatory effects of SZT-I on HFL-1 cell for eotaxin production; (H) The Log concentration-inhibition percentage curves for eotaxin inhibition of SZT-I; (I) Anti-inflammatory effects of SZT-IBu on HFL-1 cell for eotaxin production; (J) The Log concentration-inhibition percentage curves for eotaxin inhibition of SZT-IBu.

FIG. 5. Compound-Target (C-T) network of the active compounds from SZT-I for the eczema treatment. Circles and Diamonds represent compounds and targets respectively. Nodes color from blue to red are proportional to its degree. Black lines stand for interactions between nodes.

FIGS. 6A-6G. In vitro anti-TNF-α, anti-IgE effects, and cytotoxicity of active compounds. (A) anti-TNF-α effects of active compounds from SZT-I on Raw 264.7. K, Q, L stands for kaempferol, quercetin and luteolin respectively. (B) anti-TNF-α effects of luteolin on RAW 264.7 cells; (C) The IC-50 value calculated based on X=log[x] transformed data from B; (D) Luteolin Raw 264.7 cytotoxicity; (E) anti-IgE effects of luteolin on U266; (F) IC-50 value calculated based on X=log[x] transformed data from E; (G) luteolin U266 cytotoxicity. Significance is indicated as *p<0.05, **p<0.01, ***p<0.001 compared to media control.

FIGS. 7A-7D. Luteolin regulated the mRNA expression of hub targets. (A) TNF-α; (B) IL-1B; (C) Bcl2; (D) PPARG. Data are expressed the mean±error. *P<0.05, **P<0.01, ***P<0.001, ****P<0.0001.

FIG. 8. Mechanisms of SZT-I for the treatment of eczema.

FIGS. 9A-9H. Binding explorations of complex PPARG-luteolin, TNF-luteolin, IL1β-luteolin, Bcl2-luteolin. Predicted lowest-energy binding mode of luteolin in 3D- and 2D-figures with the following proteins: (A), (B) PPARG; (C), (D) TNF; (E), (F) IL1B; (G), (H) Bcl2; the carbon and oxygen are highlighted in yellow and red respectively. For residents of proteins, the green, red and blue stand for C, O and N, respectively.

FIGS. 10A-10B (A) Pathway analysis results. Y-axis: Significant 15 pathways relevant to the enriched targets; X-axis: significance of each term ranked with −log(false discovery rate) (FDR). (B) The list of genes in the 15 pathways.

FIG. 11 Compound-target-pathway network.

FIG. 12 Protein-protein interactions. The circles represent the targets. The blank lines represent the interaction between nodes. Node size is proportional to its degree in network.

FIG. 13 The 2D-HPLC fingerprint of acetonitrile extracted SZT-I. Total 26 peaks were detected and labeled.

### DETAILED DESCRIPTION

In work leading up to the present invention, the efficacy of SZT-I was verified using the Sharpin^{cpdm/cpdm} mice as eczema model. Mice with SHANK-associated RH domain interaction protein (SHARPIN) mutation (Sharpin^{cpdm/cpdm} mice) were previously shown to display the associated immunodeficiency, dermatitis symptoms, thickening skin, and multi-organ inflammation and abnormalities (Redecke et al., 2016, Immunology, 148(2), 216-226). The present inventors have shown that SZT-I treatment significantly improved the skin condition and inhibited the inflammatory response in mice model when comparing with SZT-I treatment and sham group. The anti-inflammatory effects of SZT-I on multiple cytokines and cell types indicate that the SZT-I treatment regulates multiple pathways to inhibit inflammation. The present inventors employed computational modeling and analysis to explore the active compounds and potential molecular mechanisms of SZT-I in treating eczema, which has led to identification of 38 active compounds, 130 biological targets, and 403 interactions. Multiple pathways including immune, inflammatory, and metabolic processes are involved, which indicates the multi-compound/multi-targeted modality of SZT-I in treating eczema. Based on network analysis, the present inventors have identified genes including Bcl2, IL6, TNF, PPARG, IL1B, NFKBIA, IL8, AHR, JUN, and PTGS2 as potential hub targets of SZT-I for the treatment of eczema. The present inventors have shown that luteolin, the active compound of SZT-I, displayed beneficial roles in eczema by promoting cell survival and reducing inflammation through the regulation of targets such as TNF, IL1B, Bcl2, and PPARG. The binding of luteolin with targets TNF, IL1B, Bcl2, and PPARG was established by molecular docking with excellent binding affinities. The findings by the present inventors provide insight into the molecular mechanisms underlying multi-targeted benefits of SZT-I in the treatment of eczema and provide basis for developing more effective TCM eczema therapies.

Accordingly, one aspect of the disclosure is directed to pharmaceutical composition for treating eczema comprising a therapeutically effective amount of at least one purified compound selected from Table 1, and a pharmaceutical carrier. In some embodiments, the at least one purified compound comprises a combination of purified compounds selected from Table 1.

By “combination” it is meant a combination of two or more compounds, e.g., 2 to 38 compounds, preferably selected from Table 1. In some embodiments, a combination includes two purified compounds. In some embodiments, a combination includes three purified compounds. In some embodiments, a combination includes four purified compounds. In some embodiments, a combination includes five purified compounds. In some embodiments, a combination includes six purified compounds. In some embodiments, a combination includes seven purified compounds. In some embodiments, a combination includes eight purified compounds. In some embodiments, a combination includes nine purified compounds. In some embodiments, a combination includes ten purified compounds. In some embodiments, a combination includes eleven purified compounds. In some embodiments, a combination includes twelve purified compounds. In some embodiments, a combination includes thirteen purified compounds. In some embodiments, a combination includes fourteen purified compounds. In some embodiments, a combination includes fifteen purified compounds.

In some embodiments, the at least one purified compound is selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol. In some embodiments, the combination of purified compounds is a combination of the fifteen compounds: quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol; or a sub-combination (e.g., 2, 3, 4, 5, 6, 7, 8, 9, 10, 11, 12, 13 or 14) of compounds selected from these fifteen compounds. In some embodiments, the at least one purified compound is selected from the top 10, top 5, or top 3 compounds of the fifteen compounds listed above, ranked based on the degree shown in FIG. 5.

In some embodiments, the pharmaceutical composition for treating eczema comprises (i) at least one purified compound selected from Table 1 formulated for oral administration, (ii) at least one purified compound selected from Table 1 formulated as a cream, and/or (iii) at least one purified compound selected from Table 1 formulated as a powder for soak.

In some embodiments, the at least one purified compound for each of (i), (ii) and (iii) is independently selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol. In some embodiments, the combination of purified compounds is a combination of the fifteen compounds: quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol; or a sub-combination (e.g., 2, 3, 4, 5, 6, 7, 8, 9, 10, 11, 12, 13 or 14) of compounds selected from these fifteen compounds.

In another aspect, methods of treating eczema are disclosed.

In a method of treating eczema in a subject comprises administering to the subject a pharmaceutical composition which comprises a therapeutically effective amount of at least one purified compound selected from Table 1, and a pharmaceutical carrier.

In some embodiments, the pharmaceutical composition is administered orally. In some embodiments, the pharmaceutical composition is administered by applying on a skin in the form of a cream. In some embodiments, the pharmaceutical composition is in the form of a dry powder and administered by applying on a skin in a soak.

The term “eczema” refers to a chronic skin disorder accompanied by pruritus, erythema, dryness, scaling, vesiculo-papular rash, and in some instances, sleeping disturbance. Eczema is the most common inflammatory skin disorder with rising global prevalence, which severely affects the life quality of patients and increases the social-economic burden.

The term “therapeutically effective amount” refers to an amount of a compound that, when administered to a subject, e.g., a human and a non-human animal including companion animals such as cats, dogs, for treating a disease, is sufficient to result in prevention or delay of onset of the disease, inhibition of the progression of the disease, and/or amelioration of symptoms of the disease. The “therapeutically effective amount” will vary depending on the compound, the severity of the disease, and the age, weight, etc., of the subject to be treated.

In some embodiments, a therapeutically effective amount or dose of an active compound disclosed herein (e.g., those in Table 1) for treating eczema in a human is 2 mg to 10 mg per day, or 3 mg to 9 mg per day, or 4 mg˜7.5 mg/day at a low dose. In some embodiments, a therapeutically effective amount or dose of an active compound disclosed herein (e.g., those in Table 1) for treating eczema in a human can be increased to 100 mg, 200 mg, 300 mg, 400 mg, 500 mg, 1 g, or up to 5 g per day. A higher dose will provide stronger effect. In some embodiments, for the subject (a human patient suffering eczema) is treated for 1 month, 2 months, 3 months, 4 months, 5 months, or 6 months, but can be longer than 6 months for severe conditions. In some embodiments, a pharmaceutical composition is administered once daily. In some embodiments, a pharmaceutical composition is administered multiple times, e.g., twice daily. In some embodiments, where pharmaceutical compositions in different formulations are applied (e.g., both oral and cream formulations), each formulation can be applied once or multiple times during the day. The skin condition can be monitored daily based on the standard scoring system of Atopic Dermatitis Index (SCORAD).

In some embodiments, a therapeutically effective amount or dose of an active compound disclosed herein (e.g., those in Table 1) for treating eczema in a canine animal (pet) is 0.5 mg to 3 mg per day, or 1 mg to 2.5 mg per day, or 1.5 mg˜2.5 mg/day at a low dose. In some embodiments, a therapeutically effective amount or dose of an active compound disclosed herein (e.g., those in Table 1) for treating eczema in a human can be increased to 30 mg, 60 mg, 90 mg, 120 mg, 150 mg, 300 mg, or up to 1.5 g.

In some embodiments, a therapeutically effective amount or dose of an active compound disclosed herein (e.g., those in Table 1) for treating eczema in a cat (pet) is 0.4 mg to 2 mg per day, or 0.6 mg to 1.6 mg per day, or 0.7 mg˜1.5 mg/day at a low dose. In some embodiments, a therapeutically effective amount or dose of an active compound disclosed herein (e.g., those in Table 1) for treating eczema in a human can be increased to 18 mg, 36 mg, 54 mg, 72 mg, 90 mg, 180 mg, or up to 900 mg per day.

The term “purified compound” refers to a compound in which unwanted impurities have been removed, to indicate, e.g., that the compound may be substantially enriched with respect to the complex context in which the compound is naturally present, such as in Chinese herbs or a crude extract thereof. A purified compound can also be produced in synthetic manner, distinct from the isolation or extraction from its natural milieu, while still having the same molecular structure. When the molecule is purified, the absolute level of purity is not critical and those skilled in the art can readily determine appropriate levels of purity. In some circumstances, the purified compound may be part of a composition (for example an extract containing other substances) or buffer system, which may contain other components. In other circumstances, the compound may be purified to essential homogeneity, for example as determined spectrophotometrically, by NMR or by chromatography (for example LC-MS). In certain embodiments, the term “purified” means: at least 90%, for example, 90% or 91% or 92% or 93% or 94% or 95% or 96% or 97% or 98% or 99% or 99.5% or 99.6% or 99.8% or 99.9% or 100% pure. Purified compounds can also be purchased from commercial sources.

The term “pharmaceutical carrier” includes any and all solvents, dispersion media, isotonic agents and the like. Except insofar as any conventional media, agent, diluent or carrier is detrimental to the recipient or to the therapeutic effectiveness of the active ingredients contained therein, its use is appropriate. The carrier can be liquid, semi-solid, e.g., pastes or cream, or solid carriers. Examples of carriers include oils, water, saline solutions, alcohol, sugar, gel, lipids, liposomes, resins, porous matrices, binders, fillers, coatings, preservatives and the like, or combinations thereof. In some embodiments, a pharmaceutical carrier is corn oil (food grade). Corn oil has been used to extract the active components from the herbal medicines.

The active ingredients (i.e., a purified compound or a combination of purified compounds) can be combined with the carrier in any convenient and practical manner, e.g., by admixture, solution, suspension, emulsification, encapsulation, absorption and the like, and can be made in formulations such as tablets, capsules, powder, syrup, suspensions that are suitable for injections, ingestions, topical administration, or the like.

In some embodiments, a pharmaceutical composition includes a combination of an internal formulation for oral ingestion and an external formation that can be applied to the skin in the form of cream or soak.

In some embodiments, an internal formulation is in a capsule or tablet form. In some embodiments, an external formulation is made in the form of a gel, a cream, or a paste, which can be applied to the skin topically. In some embodiments, an external formulation is made in the form of a powder or liquid which can be mixed with water and used during a bath or soak.

Table 1 lists the active compound information of SZT-I.

Fatty acid or fatty acid ester: FA; Polyene: P; Terpene: T; Flavone: F; Sterides: S; Isoflavone: I; Dihydroflavones: D; Lignans: L; Coumarin: C; Chalcone: Ch; Others: O.

### EXAMPLES

The present description is further illustrated by the following examples, which are only illustrative and by no means limiting. The contents of all cited references (including literature references, issued patents, and published patent applications as cited throughout this application) are hereby expressly incorporated by reference.

### Example 1: Materials and Methods

Formula Extract, Purification, and Analysis

SZT-I contains 8 different Chinese herbs: poria sclerotium (Fu Ling), tree peony root bark (Dan Pi), summer cypress fruit (Di Fu Zi), honeysuckle flower bud (Jin Yin Hua), red-root lithospermum root (Zi Cao), forsythia fruit (Lian Qiao), indigo dye extract from leaf (Qing Dai), and Chinese licorice root & rhizome (Sheng Gan Cao). The cream I includes 7 different herbs: tribulus fruit (Bai Ji Li), angelica root (Dang Gui), honeysuckle flower (Jin Yin Hua), lithospermum root (Zi Cao), shrubby sophora (Ku Shen), indigo dye extract from leaf (Qing Dai), and borneol (Bing Pian).

5 g of SZT-I were extracted using butanol (300 mL) and deionized water (100 mL). The butanol component, labeled as SZT-IBu, was separated and dried with a 29.53% yield. High performance liquid chromatography (HPLC) was performed using the Waters 2695 HPLC system with analytical columns (Zorbax 5 μm Sβ-C18 80A, LC 26 Column 250×4.6 mm) as previously described (Yang, et al., 2013, Phytochemistry, 95, 259-267).

Animal Studies: Eczema Model

Sharpin knockout (KO) offspring (Sharpin^{cpdm/cpdm}) were bred from heterozygous Sharpin^{cpdm/cpdm} mice, which were identified and selected through genotyping. 15 Sharpin KO mice were randomly assigned to treated, and sham groups. The treatment continued for 10 days. For the treatment group, 26 mg SZT-I was gavaged to mice, followed by dermal topical administration of cream I on the affected skin, once a day. Sham mice were topically treated with corn oil. The skin condition score, the percent area of the skin lesions, and scratching score were recorded during the experiment. The mice were sacrificed on day 12 and 13 to collect organ samples.

Scoring Atopic Dermatitis in Mice

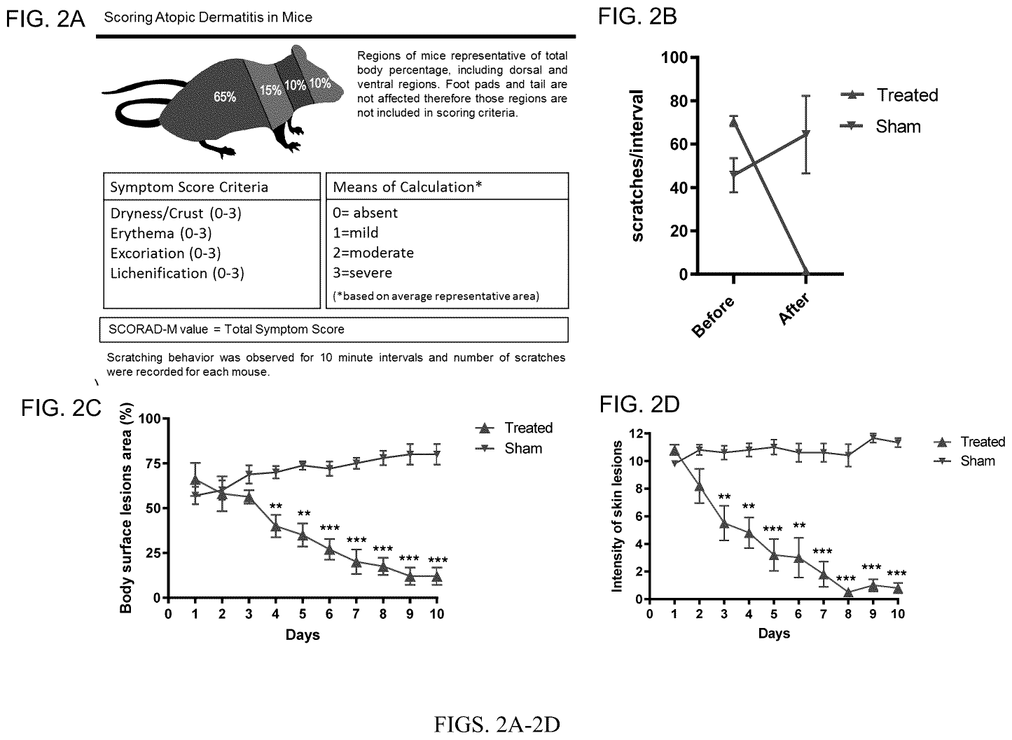

A quantitative scoring system for the severity of Atopic Dermatitis on mouse based on the SCORAD (SCORing Atopic Dermatitis) index was used on human and other eczema models published (Celakovski & Bukac, 2013, Indian J Dermatol, 58(3), 247; Murota et al., 2010, Int Arch Allergy Immunol, 153(2), 121-132). The severity of eczema in Sharpin^{cpdm/cpdm} was qualitatively evaluated. Scores were estimated based on the severity of symptoms including dryness, erythema (redness), excoriation (damaged, irritated lesion), and lichenification (thickened, leathery quality of the skin), given a scale from 0 to 3 (0: absent; 1: mild; 2: moderate; 3: severe). In addition, skin lesions were observed and photographed/recorded carefully, where area percent of lesions from the whole body were evaluated as follows: head, face, and ears: 10%; neck and back of neck: 10%; shoulders and fore-limbs: 15%; back and hind-limbs: 65% (FIG. 2A). Moreover, scratching score was evaluated based on the times which mice scratched in 10-minute intervals.

Genotyping

DNA samples of offspring were extracted from ear samples of mice. DNA was amplified by PCR with primers oligo M-RA65 fwd (CTAGGACTGTTAGGCACCGAGCCTGGC) (SEQ ID NO: 1) and oligo M-RA66 rev (CCACTTTTACAGGCAATCAAGAAAAATAACTGC) (SEQ ID NO: 2). 3% agarose gels were used to identify the size of Sharpen genes, which identified the mouse genotype.

Organ Histology and Inflammatory Cell Counts

After sacrifice, skin and liver samples of mice were fixed in 10% formalin solution and prepared in paraffin wax. Then, the samples were stained using hematoxylin and eosin (H&E) for further analysis of inflammatory cells. Sections of skin and liver were examined, and ten random foci were selected from low power, and were analyzed for eosinophil numbers by counting eosinophils per viewing field.

Computational Analysis-Target Mining

The active compounds of herbs in SZT-I were chosen from TCM Systems Pharmacology (TCMSP) database (Ru et al., 2014, Journal of Cheminformatics, 6(1), 13). Compounds with oral bioavailability (OB) over 30% and drug-likeness (DL) over 0.18 were chosen as potentially active compounds. The biological targets of compounds were identified from published databases including, Swiss Target Prediction (probability>0.5) (Daina et al., 2019, Nucleic Acids Res, 47(W1), W357-w364.; Swiss Target Prediction), Similarity Ensemble Approach (z-score>57) (Keiser et al., 2007, Nat Biotechnol, 25(2), 197-206; Similarity Ensemble Approach), PubChem (Kim et al., 2019, Nucleic Acids Res, 47(D1), D1102-d1109; PubChem) and DrugBank (DrugBank, Retrieved 10 April, 2020 from drugbank.ca; Wishart et al., 2008, Nucleic Acids Res, 36(Database issue), D901-906). All identified biological targets of compounds were mined in prevailing databases (Therapeutic Target Database (Therapeutic target database; Y. Wang et al., 2021, Ann Allergy Asthma Immunol, 126, 639-654), Genetic Association Database (Genetic association database; Pinero et al., 2020, Nucleic Acids Res, 48(D1), D845-d855), GeneCards (GeneCards; Safran et al., 2002, Bioinformatics, 18(11), 1542-1543), and Open Targets Platform (Carvalho-Silva et al., 2019; Open Targets Platform, Nucleic Acids Res, 47(D1), D1056-d1065) to select overlapped targets for eczema. Selected targets were searched in UniProt Database (uniprot.org/) (Consortium, 2018) to normalize.

Computational Analysis-Compound-Target network analysis

Compound-Target (C-T) network including potential compounds and targets of SZT-I in treating eczema were prepared by Cytoscape (v3.2.1). The properties of these two networks were analyzed by NetworkAnalyzer (Assenov et al., 2007, Bioinformatics, 24(2), 282-284.), a plugin of Cytoscape.

Computational Analysis-Gene ontology (GO), KEGG pathway analysis

To guarantee the reliability of the analysis, targets with degrees equal to or greater than 2 were selected as the main targets. The GO was conducted by searching main targets in the DAVID database (DAVID; Huang et al., 2008, Nucleic Acids Research, 37(1), 1-13), while KEGG Pathways were obtained from KOBAS 3.0 (KOBAS 3.0; Xie et al., 2011, Nucleic Acids Research, 39(supp1_2), W316-W322). GO biological process terms and significant pathways were selected with a false discovery rate of (FDR)<0.01.

Computational Analysis-Molecular docking analysis

To further investigate the interactions between active compounds and molecular targets, molecular docking was conducted by AutoDock Vina(Trott & Olson, 2010, Journal of computational chemistry, 31(2), 455-461). Protein structures including TNF (PDB: 2az5), IL1B (PDB: 5r85), Bcl2 (PDB: 5jsn), PPARG (PDB: 2prg) and compound structure were directly obtained from RCSB protein data bank (rcsb.org/) (Berman et al., 2000, Nucleic Acids Research, 28(1), 235-242) and PubChem (pubchem.ncbi.nlm.nih.gov/) (S. Kim et al., 2018, Allergy Rhinol (Providence), 9, 2152656718764145), respectively. Formats of compounds and protein were prepared through AutoDockTools (v1.5.6) (Sanner, 1999, Journal of molecular graphics & modelling, 17(1), 57-61). Graphics of conformations were illustrated by PyMOL (DeLano, 2002) (pymol.org) and Discovery Studio (DassaultSystemesBIOVIAD, 2020, Discovery Studio.

In (Version 2020)).

In vitro studies-Cell culture

Human U266 myeloma cells were cultured in complete media containing RPMI-1640 medium with 10% fetal bovine serum (FBS), 0.5% penicillin-streptomycin, 1×10⁻⁵ mol/L of 0-mercaptoethanol, and 1 mmol/L of sodium pyruvate at 37° C. in an incubator with 5% CO₂. U266 cells were cultured at initial 2×10⁵ cell/mL with SZT-I or active compounds at different concentration. Then the supernatants were collected after 6 days. The level of IgE was. measured by an enzyme-linked immunosorbent assay (ELISA) kit according to the manufacturer's instructions. RAW 264.7 murine macrophages (5×10⁵ cell/mL) were cultured in DMEM medium supplemented with 10% FBS, and 0.5% penicillin-streptomycin at 37° C. under 5% CO₂. SZT-I or active compounds at different concentrations and LPS (0.5 μg/mL) added to cells. Supernatants were harvested after 24 h and analyzed by ELISA for TNF-α (Mabtech, In vitro studies-Cell viability Inc, Cincinnati, Ohio). Eotaxin inhibitory assays were conducted by using human fetal lung fibroblast (HFL-1) cells. HFL-1 (CCL-153) cells were grown in F-12K medium (ATCC, Rockville, MD) containing 10% fetal bovine serum (Gibco BRL, Grand Island, NY) and 1% penicillin-streptomycin (BD Bio, San Jose, CA). Cells were detached using trypsin-ethylenediaminetetraacetic acid (EDTA) and transferred to 24-well culture plates, 8×104 cells per well. After 48 h, the growth medium was replaced with medium containing SZT-I or the butanol extract of SZT-I (SZT-IBu) at different concentrations. Supernatants were harvested at 96 h after the addition of the testing treatments. Eotaxin levels were determined by using ELISA (R&D Systems) according to the directions of the manufacturer.

In Vitro Studies-Cell Viability

Cell viability of U266 was measured by trypan blue exclusion (Yang, Patil, et al., 2013). Cell suspension (10 uL) was mixed with trypan blue dye (10 uL), and viable cells and total cells counted with a hemocytometer. The cell viability was calculated using the equation: cell viability=(total number of viable cells)/(total number of cells). The viability of Raw 264.7 was evaluated using the MTT assay carried out as previously described (Liu, C. et al., 2015, Phytother Res, 29(6), 925-932). Cells were seeded into a 96-well plate at 2×10⁴ cells/well, then different concentrations of SZT-I or active compounds were added to the plate. After 24 h, 50 μL MTT (2.0 mg/mL) was added to wells and the plate was incubated for a further 3 h. The supernatant was discarded, and the generated formazan was dissolved in dimethyl sulfoxide (DMSO) (200 μL). The absorbance of wells was detected at 590 nm.

In vitro studies-Real-Time Polymerase Chain Reaction (RT-PCR)

Expressions of targets were measured by RT-PCR. After treatment, total RNA was collected from the esophagus. Reverse transcription was conducted to obtain the cDNA. The RT-PCR amplification was performed using SYBRTM Green Master Mix with primers. The primer sequences were listed as follows: m-TNF-α: F-GGTGCCTATGTCTCAGCCTCTT (SEQ ID NO: 3); R-GCCATAGAACTGATGAGAGGGAG (SEQ ID NO: 4). m-Bcl2: F-CCTGTGGATGACTGAGTACCTG (SEQ ID NO: 5); R-AGCCAGGAGAAATCAAACAGAGG (SEQ ID NO: 6). m-IL1B: F-TGGACCTTCCAGGATGAGGACA (SEQ ID NO: 7); R-GTTCATCTCGGAGCC TGTGTG (SEQ ID NO: 8). m-PPARG: F-TTTCAAGGGTGCCAGTTT (SEQ ID NO: 9); R-GAGGCCAGCATCGTGTAG (SEQ ID NO: 10).

### Example 2

Treatment of Sharpin KO Mice Model Demonstrates Excellent Efficacy of SZT-I in Relieving the Symptoms of Eczema

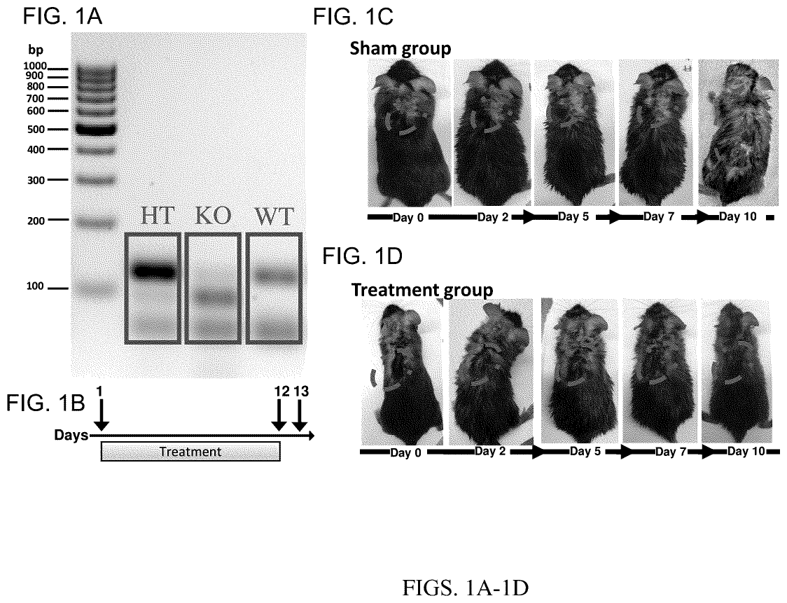

Eczema symptoms were observed in Sharpin KO mice after 6 weeks. The genotypes of offspring bred from heterozygous Sharpin^{cpdm/WT} mice were identified by PCR and agarose gel electrophoresis. Only mice with mutation Sharpin^{cpdm/cpdm} on site at CG\hat{ }CG, recognized and cut by restriction enzyme BstUI, were selected as showed in FIG. 1A. During the 10-day treatment (FIG. 1B), the representative picture of mice from the sham group (FIG. 1C) and treated group (FIG. 1D) displayed the eczema progression. Lesions of Sharpin KO mice near the neck and back of mice were exacerbated past 6 weeks.

The skin lesion of mice from sham group displayed desquamation, redness, and lichenification, while the SZT-I treated group showed significant improvement over 10-days treatment with redness, dryness and lichenification of skin dramatically reducing. The eczema symptom scores including scratches score, lesion area score, and skin condition score were recorded every day and displayed in FIG. 2. For scratches score, average scratch numbers during 10-time intervals of the sham group showed a mild increase and were still at a high level (FIG. 2B). However, the treatment group displayed a dramatic decline compared with the sham groups. The body surface lesion area showed a similar trend with scratches score, where the treatment group significantly decreased compared to the sham groups (p<0.001), and the sham group showed a mild increase during the treatment (FIG. 2C). Additionally, the intensity of skin lesion score of mice was high on day 1, but the treatment group significantly decreased to normal levels on day 10, compared with the sham group (p<0.001) (FIG. 2D). The sham group kept at a high skin lesion score with no significant difference (p>0.05). Thus, SZT-I treatment significantly improved the skin condition of eczema in mice.

### Example 3

Organ Histology and Eosinophils Counts Reveal that SZT-I Significantly Inhibit the Eosinophils Infiltration

To further verify the effect of SZT-I, organ histology and eosinophils were employed to uncover the infiltration of eosinophils. Eosinophils, as multifunctional leukocytes, are involved in various inflammatory responses and infiltrates in diverse tissues in inflammatory disorders.

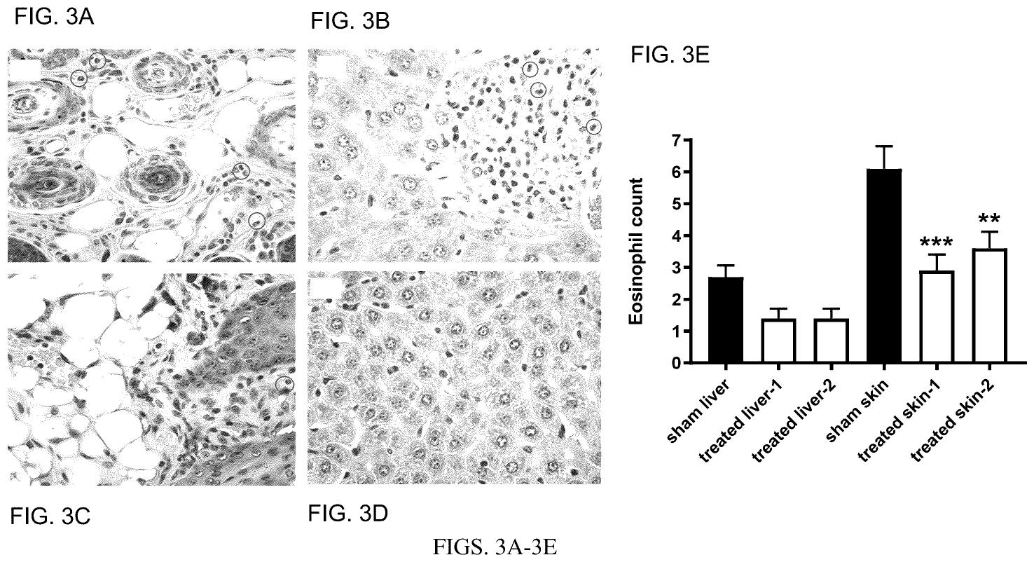

To reveal histological change of mice after treatment, the eosinophils in organ samples, including liver and skin, were further analyzed by H&E staining. The proximity of eosinophils to blood vessels reveals their infiltration into the skin, which was shown in FIG. 3A (sham group) and FIG. 3C (treatment groups). Compared with the sham group, the recruitment and infiltration of eosinophils to blood vessels dramatically declined after treatment. In addition, the liver samples from the sham group (FIG. 3B) and treatment group (FIG. 3D) revealed the significant improvement of eosinophil infiltration in the liver. The number of eosinophils in liver and skin samples are shown in FIG. 3E. For skin samples, the eosinophils count in both treated groups were significantly decreased (p<0.01, p<0.001) compared with sham group. In addition, a notable decrease was detected although no significant changes (p>0.05) were found comparing the treated groups with sham group in liver samples. The results reveal that SZT-I significantly inhibits the eosinophils infiltration, which might contribute to the improvement of symptoms in the skin.

### Example 4

Significant Anti-TNF-α, Anti-IgE, and Anti-Eotaxin Effects of SZT-I

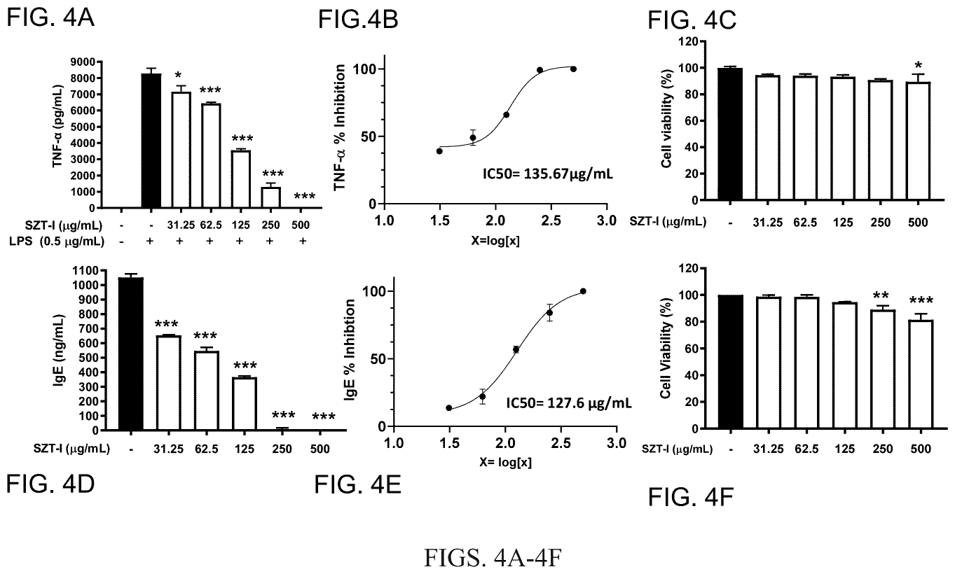

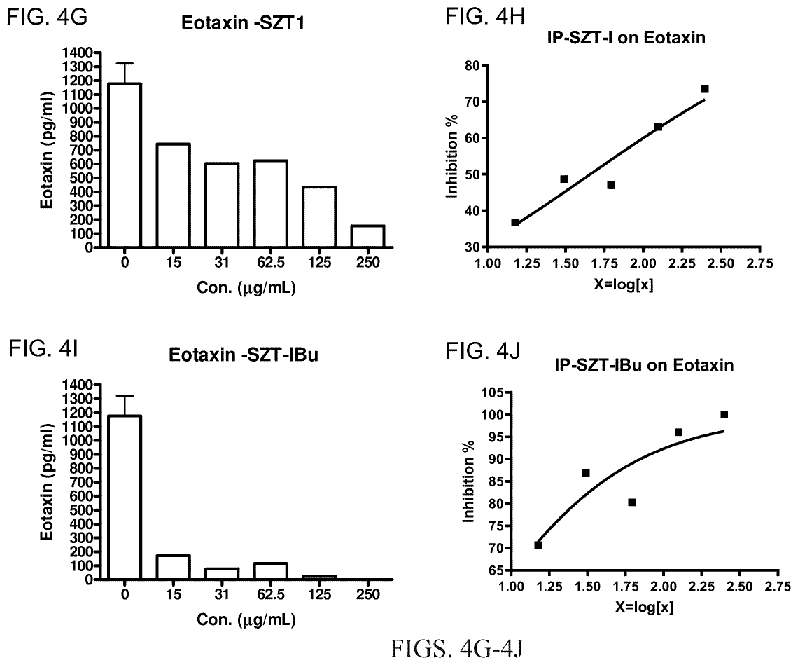

Inflammatory and immune responses displayed important roles in the development of eczema. The anti-inflammatory effect was further evaluated by in vitro cell model. As demonstrated in FIG. 4, SZT-I extracts significantly inhibited the production of TNF-α by Raw 246.7 treated with LPS (FIG. 4A). The IC₅₀ (half-maximal inhibitory concentration) is approximately 135.67 μg/mL (FIG. 4B). SZT-I extracts showed no significant cell toxicity below concentration 250 μg/mL (FIG. 4C). The inhibition of SZT-I on IgE production was further evaluated by the U266 cell model. Even at low concentrations (31.25 μg/mL and 62.5 g/mL), SZT-I significantly reduced the production of IgE, without cytotoxicity (FIG. 4D, 4F). The IC₅₀ is approximately 127.6 μg/mL (FIG. 4E). The results indicate that SZT-I significantly suppressed the production of inflammatory factors TNF-α and IgE, possibly contributing to the treatment of eczema. To determine the potential anti-inflammatory effects of the SZT-I formula and the butanol extract SZT-I formula (SZT-IBu), we tested the effects of these two formulae on HFL-1 cell for eotaxin production. Both formulas inhibited the eotaxin production dose-dependently (FIGS. 4G and I). The Log concentration-inhibition percentage curves for eotaxin inhibition was generated using Prism software (FIGS. 4H and J). The SZT-IBu showed more potency of inhibitory effect on eotaxin production than SZT-I. The IC50 value of SZT-IBu is 4.98 μg/mL, while the SZT-I showed IC50 value of 45.54 μg/mL.

### Example 5

Computational Analysis Uncovered the Targets and Mechanisms of SZT-I in Treating Eczema

Recently, with the development of cheminformatics and bioinformatics, computational methods such as network pharmacology and molecular docking have been used as important tools to explore the complex mechanisms of TCM formula. According to the principle of absorption, distribution, metabolism, and excretion analysis, in total 187 active compounds (OB;>30%, DL>0.18) from the formula of SZT-I were collected from TCM Systems Pharmacology (TCMSP). Of which, 23 were from Lian Qiao (LQ), 92 from Gan Cao (GC), 15 from Fu Ling (FL), 11 from Dan Pi (DP), 2 from Di Fu Zi (DFZ), 23 from Jin Yin Hua (JYH), 12 from Zi Cao (ZC), and 9 from Qing Dai (QD). Further mapping the targets of these potentially active compounds into eczema biological targets, 38 compounds were found to share 130 biological targets with eczema, which might be potential targets of SZT-I for treating eczema. Information including compound name, molecular weight, and structure are listed in Table 1. Among 38 active compounds, flavonoid kaempferol (OB=41.88%, DL=0.24) and quercetin (OB=46.43%, DL=0.28) were found from both Lian Qiao, Dan Ping, Gan Cao and Jin Yin Hua, and luteolin (OB=36.16%, DL=0.25) was found in both Lian Qiao and Jin Yin Hua. Moreover, phytosterols including β-sitosterol (OB=36.91%, DL=0.75), sitosterol (OB=36.91%, DL=0.75), and stigmasterol (OB=43.83, DL=0.76) were also mutual ingredients shared by more than one herb. Surprisingly, all 6 mutual compounds have been reported to display remarkable pharmacological effects. Flavonoid kaempferol, quercetin, and Luteolin can inhibit the production of pro-inflammatory cytokines and improve Th1/Th2 balance (Mlcek et al., 2016; Molecules (Basel, Switzerland), 21(5), 623; Park & Song, 2013, Nutr Res Pract, 7(6), 423-429). Phytosterols such as β-sitosterol were proven to improve Th1 cells and inhibit Th2 cells as immune-regulator (Bouic & Lamprecht, 1999, Altern Med Rev, 4(3), 170-177). Thus, phytosterols has drawn increased interest as alternative medicine for the treatment of eczema (Kim, 2017, Biomed Sci Letters, 23, 303-309).

Compound-Target (C-T) network involving 38 compounds, 130 targets, and 403 interactions were constructed as FIG. 5, where circles and diamonds represented active compounds and interrelated targets, respectively. The network indicates multi-interactions between compounds and targets. The majority of compounds may affect more than one protein target to intervene in the progression of eczema. Similarly, the majority of targets of eczema can be regulated by more than one ingredient in the formula, which further implies the synergistic effects of TCM. The influence of nodes in the network can be evaluated by one of the parameters “degree”. The node degree is calculated by the number of connected edges, representing the number of interactions of nodes. The network average degree of node was 4.8.

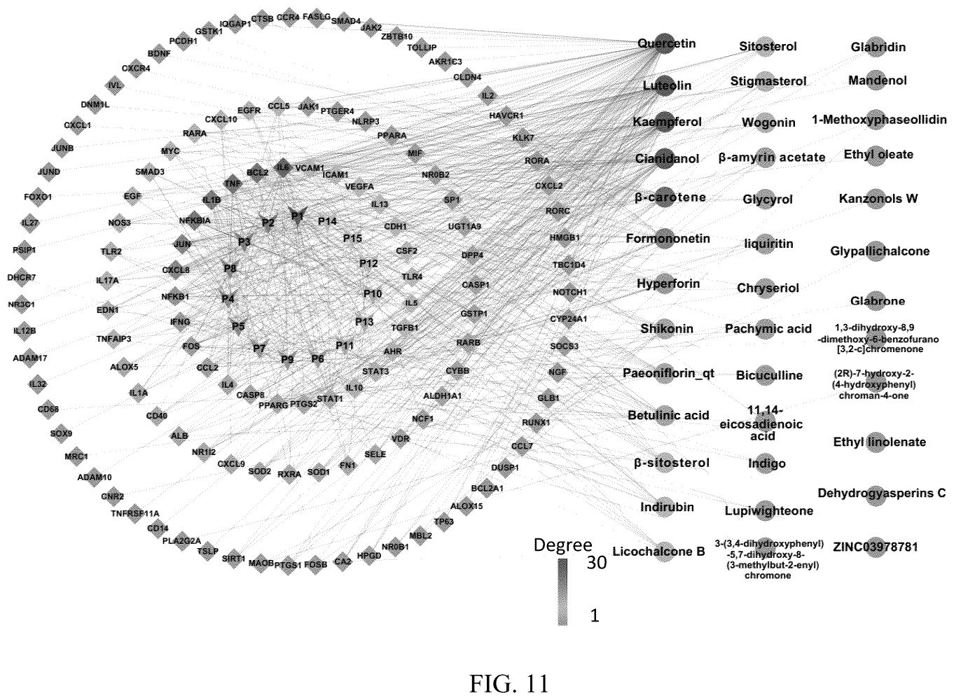

Among the 38 compounds, the degree of quercetin, luteolin, and kaempferol is 74, 50, 48 respectively, which were ranked as the top three hub ingredients, indicating their essential roles in the pharmacology of SZT-I. Based on the degree rank, the top ten hub targets are as follows: Bcl2 (16), IL6 (14), TNF (13), PPARG (12), IL1B (12), NFKBIA (11), CXCL8 (10), AHR (10), JUN (9), and PTGS2 (9). The hub targets of C-T network are similarly prominent in the protein-protein interaction (PPI) network (FIG. 11), indicating the predominant roles of such targets in overall regulation.

### Example 6

Pathway Analysis and Compound-Target-Pathway Network Revealed the Regulation of SZT-I in the Inflammatory and Immune Processes

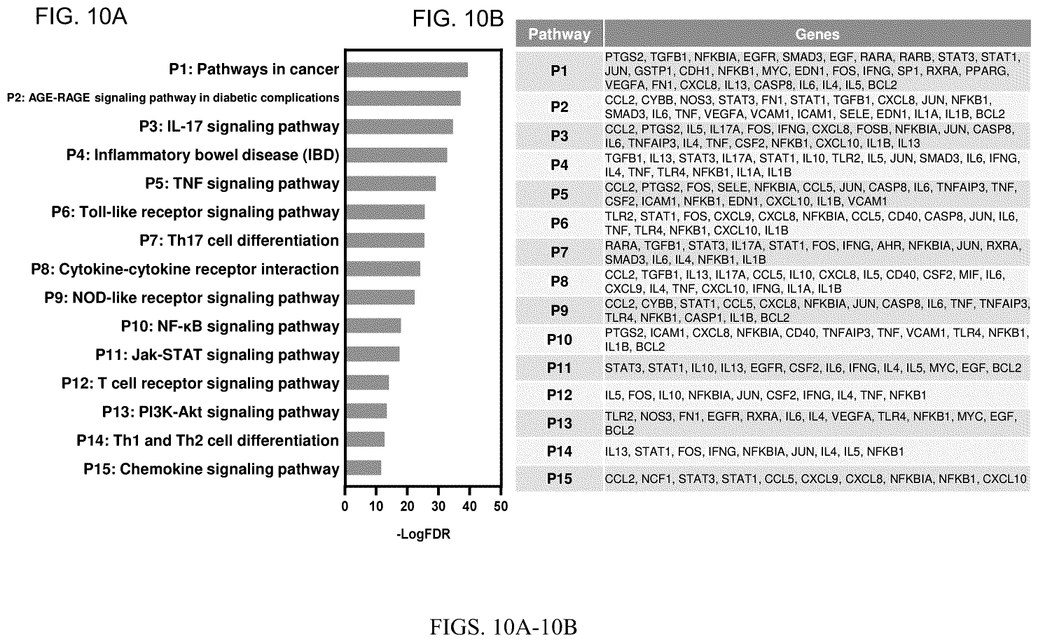

Among 130 targets of SZT-I, 74 targets were regulated by more than 1 compound. To increase the credibility of the analysis, 74 targets were employed to conduct gene-set enrichment analysis in KOBAS database. The 15 closely related pathways are ranked by enrichment score (−logFDR) in FIG. 10A. Pathways including IL-17 signalling pathway (P3), Th17 cell differentiation (P7), NOD-like receptor signalling pathway (P9), T cell receptor signalling pathway (P12), and Th1 and Th2 cell differentiation (P14) are related to the immune responsive processes protecting against the infection of pathogens. Inflammatory pathways such as Inflammatory bowel disease (IBD) (P4), TNF signalling pathway (P5), and NF-1B signalling pathway (P10) represented key regulation of inflammatory reactions. Upstream pathways such as the pathway in cancer (P1), Jak-STAT signalling pathway (P11), and PI3K-Akt signalling pathway (P13) play important roles in various biological processes, such as cell proliferation, differentiation, and immune reaction. By regulating these crucial targets and pathways, SZT-I may act on the regulation of immune, inflammatory, and metabolic processes, but not limited to the above pathways. The genes involved in the 15 pathways were listed in FIG. 10B. The compound-target-pathway (C-T-P) network (FIG. 11) was extracted as the key regulated network of SZT-I. Hub targets determined by C-β-T network were consistent with the C-T network, which further confirmed the significance of targets and pathways in the network.

### Example 7

Protein-Protein Interaction (PPI) Network Further Confirm the Vital Function of Hub Targets

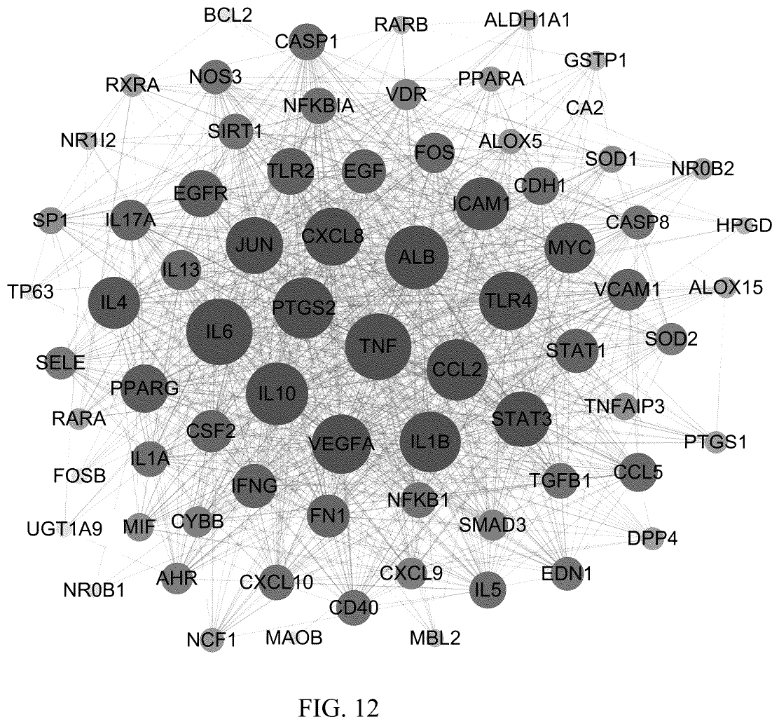

The PPI network (FIG. 12) was prepared in the String database, which is necessary to exhibit the multi-interaction between targets in cellular processes. The nodes in larger size and in the center position stands for the important targets in the network. As expected, hub targets such as TNF, IL6, IL8, PPARG, and IL1B displayed the most interactions with other proteins. Moreover, TLR4, as an important toll-like receptor, plays a key role in LPS recognition and LPS-mediated inflammatory response (Plóciennikowska et al., 2015, Cell Mol Life Sci, 72(3), 557-581). STAT1 and STAT3 belong to the family of nuclear transcription factors, which regulated various downstream genes involved in the cell cycle, cell survival, and immune response (Butturini et al., 2020, Signaling. IntJMol Sci, 21(19)).

### Example 8

HPLC Fingerprint of Acetonitrile Extracted SZT-I

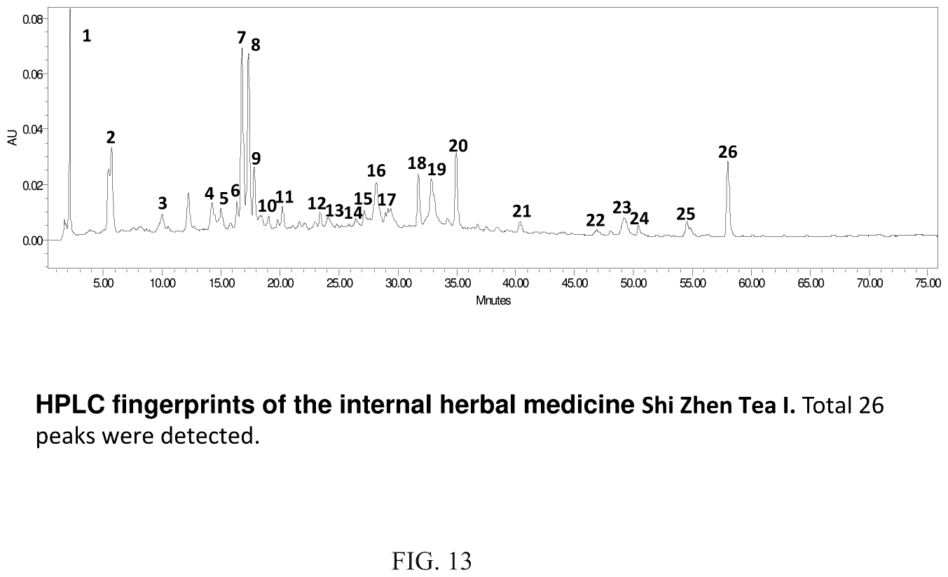

The 2D-HPLC fingerprint of acetonitrile extracted SZT-I formula was generated at 254 nm (FIG. 13). Total 26 peaks were detected and labeled in the FIG. 13.

### Example 9

In Vitro Anti-TNF-α, and Anti-IgE Effects of Active Compounds Disclosed the Anti-Inflammation and Anti-Allergy of SZT-I

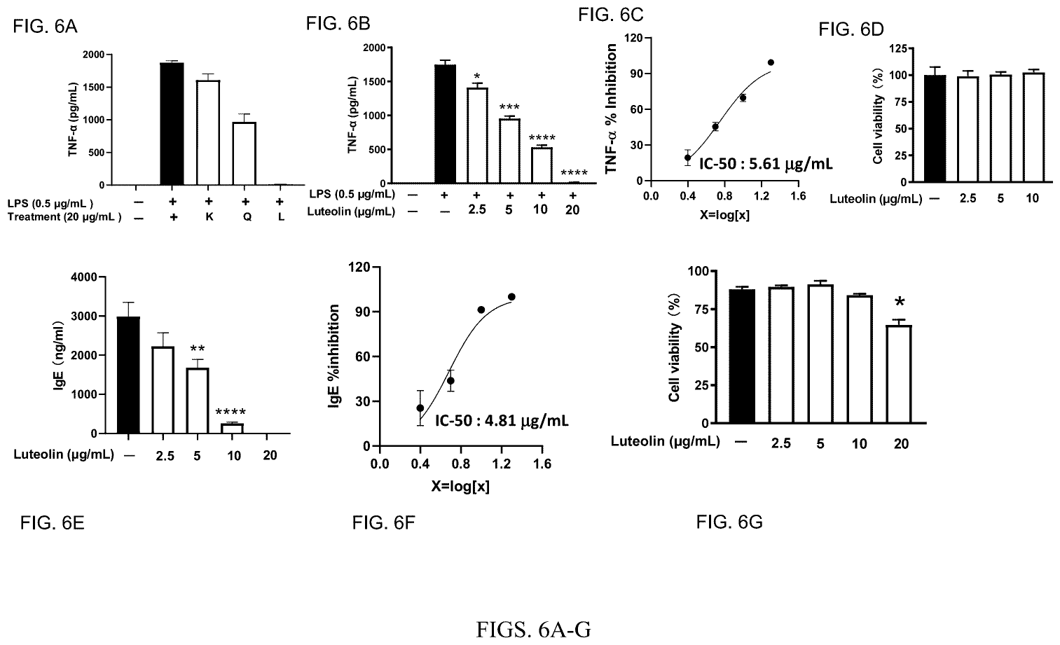

Through computational analysis of SZT-I, the top-3 compounds were chosen with the most targets (quercetin, luteolin, and kaempferol) as active compounds. At first, the anti-TNF effects were verified on Raw 264.7 cells stimulated by LPS. As of FIG. 6A, luteolin significantly inhibited the production of TNF-α compared with quercetin and kaempferol at the same concentration (20 μg/mL). Thus, luteolin was considered as the main active compound to further validate its pharmacological activities. With the increase of luteolin concentration, the production of TNF-α gradually decreased (FIG. 6B) with IC₅₀ of 5.61 μg/mL (FIG. 6C). The luteolin showed no significant cell toxicity for Raw 246.7 (FIG. 6D). Ability of luteolin to inhibit the production of IgE was further evaluated on the U266 cell model. At 10 μg/mL concentration, luteolin strongly diminished production of IgE without cytotoxicity (FIG. 6E, 6G). The IC₅₀ is around 4.81 μg/mL (FIG. 6F). As an active compound of SZT-I, luteolin displayed significant anti-inflammation and anti-allergy effects through in vitro cell model.

### Example 10

Luteolin Suppressed LPS-Induced Cell Apoptosis and Inflammatory Responses Through Targets TNF, IL1B, Bcl2, and PPARG

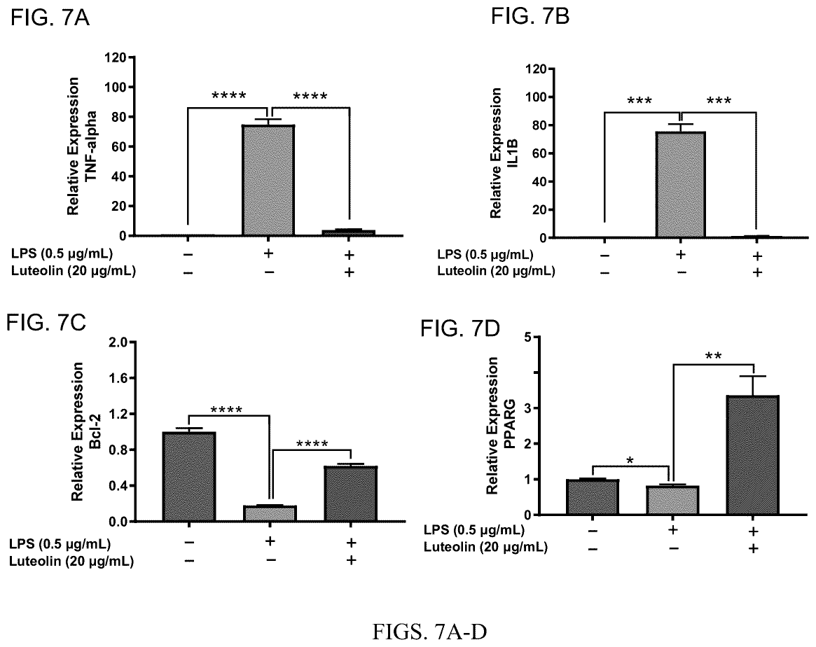

The effects of luteolin on hub targets including TNF, IL1B, Bcl2, and PPARG were further determined by PCR analysis. As showed in FIG. 7, the expression of TNF-α and IL1B increase significantly after LPS stimulation, which was further reversed by luteolin treatment, indicating the strong anti-inflammatory activities of luteolin (FIG. 7A, 7B). LPS inhibited the expression of Bcl2 in Raw 246.7 cells, and luteolin increased the expression of Bcl2 significantly (FIG. 7C), which might prevent apoptosis and lead to cell survival and proliferation. In addition, a large number of genes regulating lipid metabolism, cell proliferation, and inflammation are controlled by PPARG (Dubuquoy et al., 2006, Gut, 55(9), 1341-1349). PPARG can not only inhibit the NF-κB pathway by reacting with the inhibitory protein IKB, but also regulate the MAPK pathway to reduce the activation of JNK and p38, as well as inhibit the transcription factors such as c-Jun, c-fos (Dubuquoy et al., 2006, Gut, 55(9), 1341-1349). As one of the promising treated targets associated with the development of eczema. Luteolin upregulated the expression of PPARG inhibited by LPS (FIG. 7D), which might be associated with the down regulation of various inflammatory cytokines. As showed in FIG. 8, active compounds from SZT-I exert beneficial improvement in the skin condition of eczema in facilitating the resolution of inflammation and by promoting cell survival through regulating hub targets such as TNF, IL1B, Bcl2, and PPARG.

### Example 11

Molecular Docking Predicted the Binding Modes Between Luteolin with Targets TNF, IL1B, Bcl2, and PPARG

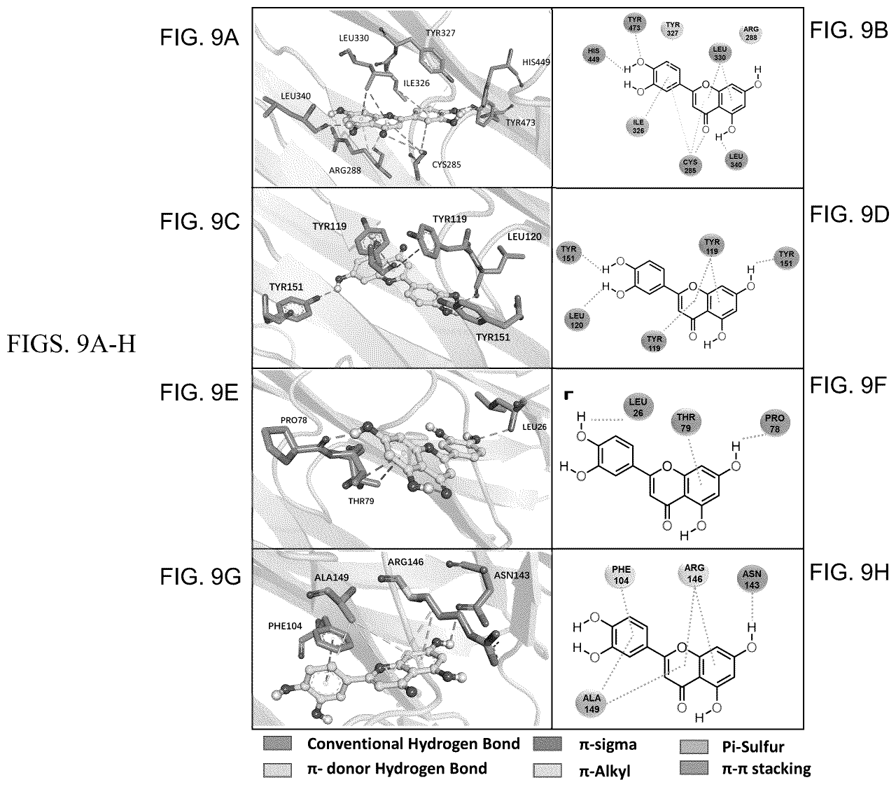

Genes including TNF, IL1B, Bcl2, and PPARG have been validated as potential targets of luteolin. Luteolin might regulate the crucial targets by interfering with genes expression or binding with proteins directly. Thus, molecular docking was used to evaluate the binding possibility between luteolin and targets. The results indicated that luteolin might be an effective inhibitor for TNF and IL1B, and a promising activator for Bcl2, and PPARG with moderate binding energy (−6.5-8.0 kcal/mol) (Table 2) (Eleftheriou et al., 2020, Virus. Molecules, 25(11).). The compound BRL are used as positive activator, which provided −8.4 kcal/mol binding energy with PPARG. The PPARG showed the highest binding affinity with luteolin. Residues TYR473, HIS449, CYS285, and LEU340 formed hydrogen bonds with luteolin (FIG. 9A, 9B). The TYR327, ARG288, LEU330 and ILE326 provided 90 interactions to further stabilize the complex. The compound 307, diacerein, and venetoclax are used as positive inhibitor for TNF, IL1B, and Bcl2. The TNF-luteolin (−7.5 kcal/mol) and IL1β-luteolin (−7.1 kcal/mol) showed moderate binding energy, among which hydrogen binding and 90 interaction are major contributions (FIG. 9C-9F). Complex Bcl2-luteolin displayed acceptable binding energy (−6.5 kcal/mol), where ASN143 provided hydrogen bonds and residues such as PHE104, ARG146 and ALA149 served π-alkyl interactions with luteolin (FIG. 9G, 9H). The molecular docking results indicated that luteolin might bind with the crucial proteins directly to regulate the following biological effects.

TABLE 2

| Gene | | |
|---|---|---|
| Name | Compounds | Affinity (Kcal/mol) |
| PPARG | Luteolin | −8.0 |
| | Positive Activator: BRL^{a} | −8.4 |
| TNF | Luteolin | −7.5 |
| | Positive Inhibitor: 307^{b} | −8.8 |
| IL1B | Luteolin | −7.1 |
| | Positive Inhibitor: Diacerein | −7.0 |
| Bcl2 | Luteolin | −6.5 |
| | Positive Inhibitor: Venetoclax | −8.8 |

^{a}BRL: 2,4-THIAZOLIDIINEDIONE, 5-[[4-[2-(METHYL-2-PYRIDINYLAMINO)ETHOXY]PHENYL]METHYL]-(9CL)^{b}307: 6,7-DIMETHYL-3-[(METHYL{2-[METHYL({1-[3-(TRIFLUOROMETHYL)PHENYL]-1H-INDOL-3 YL}METHYL)AMINO]ETHYL}AMINO)METHYL]-4H-CHROMEN-4-ONE
^{b}307: 6,7-DIMETHYL-3-[(METHYL{2-[METHYL({1-[3-(TRIFLUOROMETHYL)PHENYL]-1H-INDOL-3 YL}METHYL)AMINO]ETHYL}AMINO)METHYL]-4H-CHROMEN-4-ONE

TABLE 1

| Herb | Comp. Name | Mol. Wt. | O.B. | D.L. | Structure | Sp. |
|---|---|---|---|---|---|---|
| JinYinHua ZiCao | Mandenol | 308.56 | 42 | 0.19 | 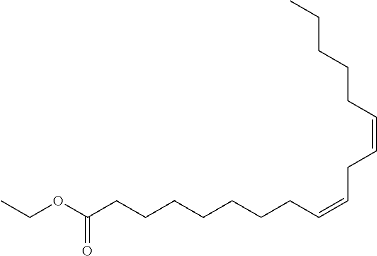 [SMILES: CCCCC/C=C\C/C=C\CCCCCCCC(=O)OCC] | FA |
| JinYinHua | Ethyl linolenate | 306.54 | 46.1 | 0.2 | 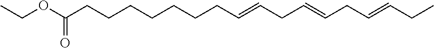 [SMILES: CC/C=C/C/C=C/C/C=C/CCCCCCCC(=O)OCC] | FA |
| JinYinHua | β-carotene | 536.96 | 37.18 | 0.58 | 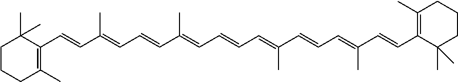 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1] | P |
| JinYinHua | ZINC03978781 | 412.77 | 43.83 | 0.76 | 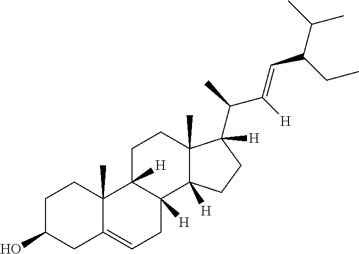 | T |
| JinYinHua | Chryseriol | 286.25 | 35.85 | 0.27 | 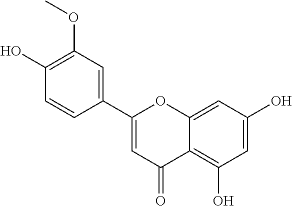 [SMILES: COc1cc(-c2cc(=O)c3c(O)cc(O)cc3o2)ccc1O] | F |
| JinYinHua QingDai LianQiao | β-sitosterol | 414.79 | 36.91 | 0.75 | 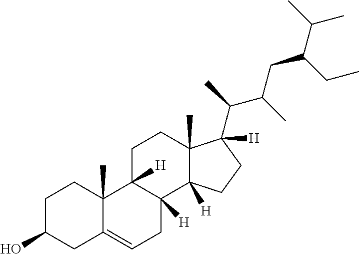 | S |
| JinYinHua DanPing GanCao | Kaempferol | 286.25 | 41.88 | 0.24 | 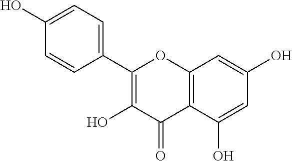 [SMILES: O=c1c(O)c(-c2ccc(O)cc2)oc2cc(O)cc(O)c12] | F |
| JinYinHua DiFuZi | Stigmasterol | 412.77 | 43.83 | 0.76 | 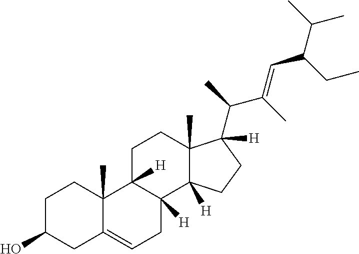 | S |
| JinYinHua LianQiao | Luteolin | 286.25 | 36.16 | 0.25 | 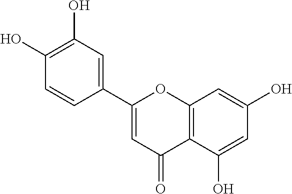 [SMILES: O=c1cc(-c2ccc(O)c(O)c2)oc2cc(O)cc(O)c12] | F |
| JinYinHua LianQiao DiPing | Quercetin | 302.25 | 46.43 | 0.28 | 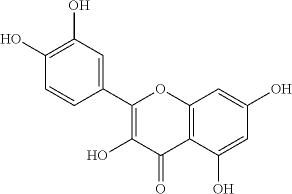 [SMILES: O=c1c(O)c(-c2ccc(O)c(O)c2)oc2cc(O)cc(O)c12] | F |
| ZiCao | Ethyl oleate | 310.58 | 32.4 | 0.19 | 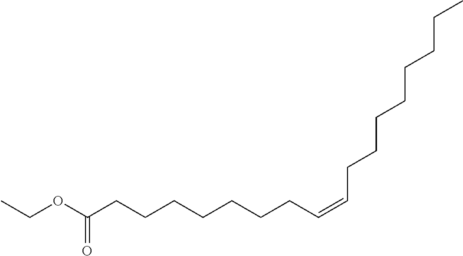 [SMILES: CCCCCCCC/C=C\CCCCCCCC(=O)OCC] | FA |
| ZiCao | Shikonin | 288.32 | 64.79 | 0.2 | 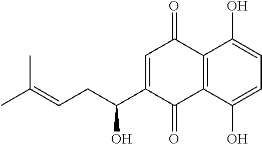 [SMILES: CC(C)=CC[C@H](O)C1=CC(=O)c2c(O)ccc(O)c2C1=O] | O |
| ZiCao DanPi ZiCao | Sitosterol | 414.79 | 36.91 | 0.75 | 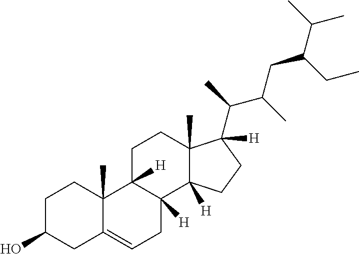 | S |
| LianQiao DanPi GanCao | Betulinic acid | 456.78 | 55.38 | 0.78 | 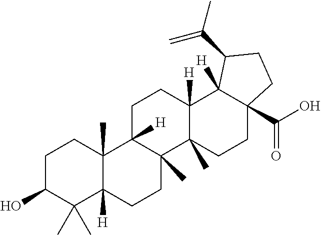 | T |
| LianQiao | β-amyrin acetate | 468.84 | 42.06 | 0.74 | 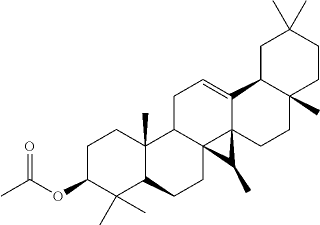 | T |
| LianQiao | hyperforin | 536.87 | 44.03 | 0.6 | 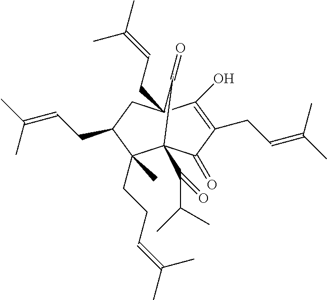 [SMILES: CC(C)=CCC[C@]1(C)[C@@H](CC=C(C)C)C[C@]2(CC=C(C)C)C(=O)[C@]1(C(=O)C(C)C)C(=O)C(CC=C(C)C)=C2O] | P |
| LianQiao | bicuculline | 367.38 | 69.67 | 0.88 | 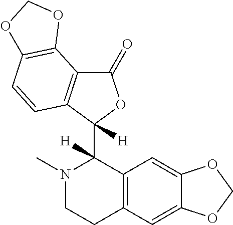 [SMILES: CN1CCc2cc3c(cc2[C@H]1[C@H]1OC(=O)c2c1ccc1c2OCO1)OCO3] | A |
| LianQiao | wogonin | 284.28 | 30.68 | 0.23 | 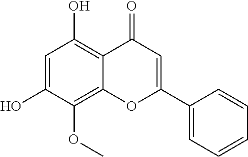 [SMILES: COc1c(O)cc(O)c2c(=O)cc(-c3ccccc3)oc12] | F |
| GanCao | Glycyrol | 366.39 | 90.78 | 0.67 | 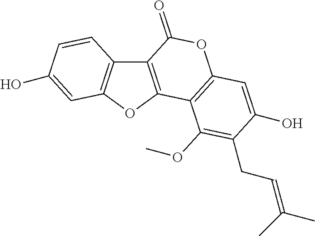 [SMILES: COc1c(CC=C(C)C)c(O)cc2oc(=O)c3c4ccc(O)cc4oc3c12] | C |
| GanCao | Lupiwighteone | 338.38 | 51.64 | 0.37 | 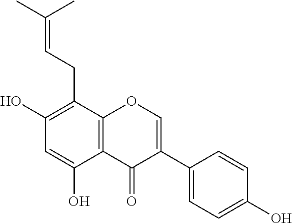 [SMILES: CC(C)=CCc1c(O)cc(O)c2c(=O)c(-c3ccc(O)cc3)coc12] | I |
| GanCao | Formononetin | 268.28 | 69.67 | 0.21 | 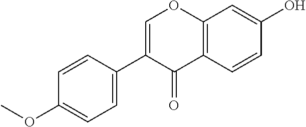 [SMILES: COc1ccc(-c2coc3cc(O)ccc3c2=O)cc1] | I |
| GanCao | Kanzonols W | 336.36 | 50.48 | 0.52 | 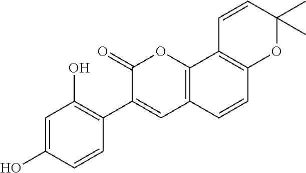 [SMILES: CC1(C)C=Cc2c(ccc3cc(-c4ccc(O)cc4O)c(=O)oc23)O1] | C |
| GanCao | Glypallichalcone | 284.33 | 61.6 | 0.19 | 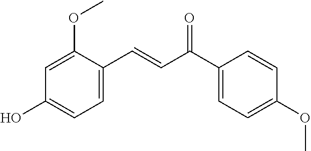 [SMILES: COc1ccc(C(=O)/C=C/c2ccc(O)cc2OC)cc1] | Ch |
| GanCao | Licochalcone B | 286.3 | 76.76 | 0.19 | 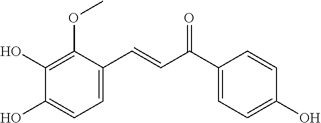 [SMILES: COc1c(/C=C/C(=O)c2ccc(O)cc2)ccc(O)c1O] | C |
| GanCao | 3-(3,4- dihydroxyphenyl)- 5,7-dihydroxy-8- (3-methylbut-2- enyl)chromone | 354.38 | 66.37 | 0.41 | 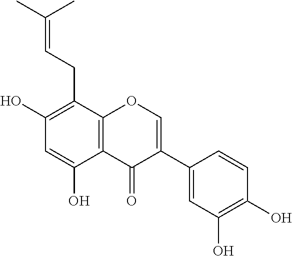 [SMILES: CC(C)=CCc1c(O)cc(O)c2c(=O)c(-c3ccc(O)c(O)c3)coc12] | I |
| GanCao | liquiritin | 410.8 | 65.69 | 0.74 | 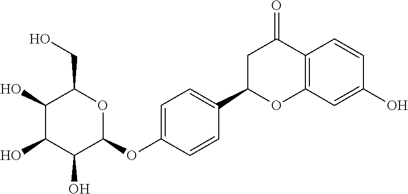 [SMILES: O=C1C[C@H](c2ccc(O[C@@H]3O[C@H](CO)[C@H](O)[C@H](O)[C@@H]3O)cc2)Oc2cc(O)ccc21] | D |
| GanCao | Glabridin | 324.4 | 53.25 | 0.47 | 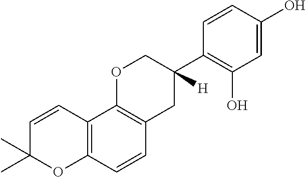 [SMILES: CC1(C)C=Cc2c(ccc3c2OC[C@H](c2ccc(O)cc2O)C3)O1] | L |
| GanCao | Glabrone | 336.36 | 52.51 | 0.5 | 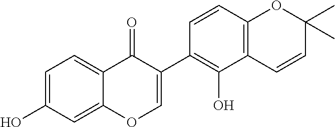 [SMILES: CC1(C)C=Cc2c(ccc(-c3coc4cc(O)ccc4c3=O)c2O)O1] | I |
| GanCao | 1,3-dihydroxy- 8,9-dimethoxy-6- benzofurano[3,2- c]chromenone | 328.29 | 62.9 | 0.53 | 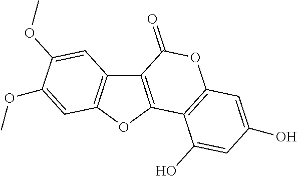 [SMILES: COc1cc2oc3c4c(O)cc(O)cc4oc(=O)c3c2cc1OC] | C |
| GanCao | (2R)-7-hydroxy- 2-(4- hydroxyphenyl) chroman-4-one | 256.27 | 71.12 | 0.18 | 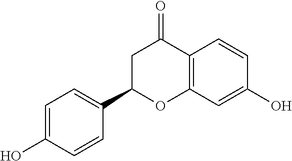 [SMILES: O=C1C[C@H](c2ccc(O)cc2)Oc2cc(O)ccc21] | D |
| GanCao | 1- Methoxyphaseol- lidin | 354.43 | 69.98 | 0.64 | 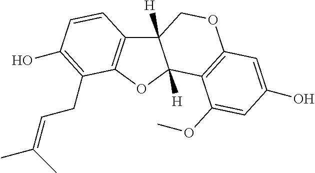 [SMILES: COc1cc(O)cc2c1[C@@H]1Oc3c(ccc(O)c3CC=C(C)C)[C@@H]1CO2] | L |
| GanCao | dehydroglyasperins C | 340.4 | 53.82 | 0.37 | 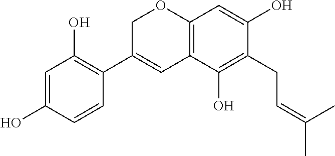 [SMILES: CC(C)=CCc1c(O)cc2c(c1O)C=C(c1ccc(O)cc1O)CO2] | L |
| DanPi | paeoniflorin_qt | 318.35 | 68.18 | 0.4 | 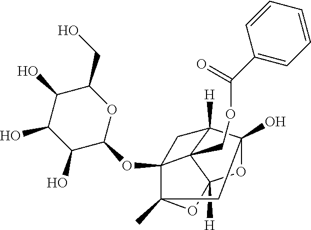 [SMILES: C[C@@]12C[C@@]3(O)O[C@H](O1)[C@@]1(COC(=O)c4ccccc4)[C@@H]3C[C@]12O[C@@H]1O[C@H](CO)[C@H](O)[C@H](O)[C@@H]1O] | O |
| DanPi | Cianidanol | 414.79 | 36.91 | 0.75 | 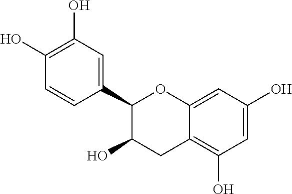 [SMILES: Oc1cc(O)c2c(c1)O[C@H](c1ccc(O)c(O)c1)[C@H](O)C2] | L |
| DiFuZi | 11,14- eicosadienoic acid | 308.56 | 39.99 | 0.2 | 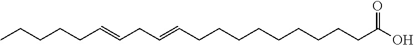 [SMILES: CCCCC/C=C/C/C=C/CCCCCCCCCC(=O)O] | FA |
| QingDai | Indigo | 262.28 | 38.2 | 0.26 | 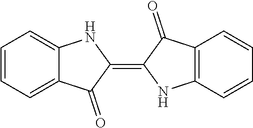 [SMILES: O=C1/C(=C2\Nc3ccccc3C2=O)Nc2ccccc21] | O |
| QingDai | indirubin | 262.28 | 48.59 | 0.26 | 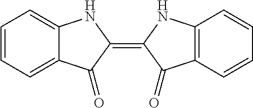 [SMILES: O=C1/C(=C2/Nc3ccccc3C2=O)Nc2ccccc21] | O |
| FuLing | Pachymic acid | 528.85 | 33.63 | 0.81 | 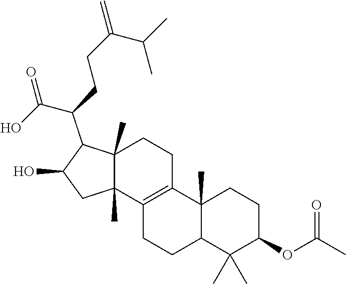 [SMILES: C=C(CC[C@H](C(=O)O)C1[C@H](O)C[C@@]2(C)C3=C(CC[C@@]12C)[C@]1(C)CC[C@@H](OC(C)=O)C(C)(C)C1CC3)C(C)C] | T |

What is claimed is:

1. A pharmaceutical composition for treating eczema, comprising
a therapeutically effective amount of at least one purified compound selected from Table 1, and
a pharmaceutical carrier.

2. The pharmaceutical composition of claim 1, wherein said at least one purified compound comprises a combination of purified compounds selected from Table 1.

3. The pharmaceutical composition of claim 2, wherein said combination of purified compounds selected from Table 1 comprises at least two purified compounds selected from Table 1.

4. The pharmaceutical composition of claim 2, wherein said combination of purified compounds selected from Table 1 comprises at least three purified compounds selected from Table 1.

5. The pharmaceutical composition of claim 2, wherein said combination of purified compounds selected from Table 1 comprises at least four purified compounds selected from Table 1.

6. The pharmaceutical composition of claim 1, wherein said at least one purified compound is selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

7. The pharmaceutical composition of claim 1, wherein said at least one purified compound is selected from quercetin, luteolin or kaempferol.

8. The pharmaceutical composition of claim 1, wherein said at least one purified compound is luteolin.

9. The pharmaceutical composition of claim 1, wherein said at least one purified compound comprises a combination of purified compounds selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

10. The pharmaceutical composition of claim 1, wherein said at least one purified compound comprises a combination of purified compounds selected from quercetin, luteolin or kaempferol.

11. The pharmaceutical composition of claim 10, wherein the combination of purified compounds includes luteolin.

12. The pharmaceutical composition of claim 1, wherein the pharmaceutical composition is formulated for oral administration.

13. The pharmaceutical composition of claim 1, wherein the pharmaceutical composition is formulated as a cream.

14. The pharmaceutical composition of claim 1, wherein the pharmaceutical composition is in the form of a dry powder.

15. A pharmaceutical composition for treating eczema comprising
(i) at least one purified compound selected from Table 1 formulated for oral administration,
(ii) at least one purified compound selected from Table 1 formulated as a cream, and/or
(iii) at least one purified compound selected from Table 1 formulated as a powder for soak.

16. The pharmaceutical composition of claim 15, wherein said at least one purified compound comprises a combination of purified compounds selected from Table 1.

17. The pharmaceutical composition of claim 15, wherein said at least one purified compound for each of (i), (ii) and (iii) is independently selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

18. The pharmaceutical composition of claim 15, wherein said at least one purified compound for each of (i), (ii) and (iii) is independently selected from quercetin, luteolin or kaempferol.

19. The pharmaceutical composition of claim 15, wherein said at least one purified compound for each of (i), (ii) and (iii) is luteolin.

20. A method of treating eczema in a subject comprising administering to the subject a pharmaceutical composition which comprises
a therapeutically effective amount of at least one purified compound selected from Table 1, and
a pharmaceutical carrier.

21. The method of claim 20, wherein said at least one purified compound comprises a combination of purified compounds selected from Table 1.

22. The method of claim 20, wherein said at least one purified compound is selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

23. The method of claim 20, wherein said at least one purified compound is quercetin, luteolin or kaempferol.

24. The method of claim 20, wherein said at least one purified compound is luteolin.

25. The method of claim 20, wherein the pharmaceutical composition is administered orally.

26. The method of claim 20, wherein the pharmaceutical composition is administered by applying on a skin in the form of a cream.

27. The method of claim 20, wherein the pharmaceutical composition is in the form of a dry powder and administered by applying on a skin in a soak.

28. A method of treating eczema in a subject comprising administering to the subject
(i) at least one purified compound selected from Table 1 via oral administration,
(ii) at least one purified compound selected from Table 1 via topical administration as a cream, and/or
(iii) at least one purified compound selected from Table 1 as a powder for soak.

29. The method of claim 28, wherein said at least one purified compound comprises a combination of purified compounds selected from Table 1.

30. The method of claim 28, wherein said at least one purified compound for each of (i), (ii) and (iii) is independently selected from the group consisting of quercetin, luteolin, kaempferol, catechin, β-carotene, formononetin, wogonin, paeoniflorin qt, betulinic acid (mairin), isoarnebrin 4, β-sitosterol, indirubin, sitosterol, licochalcone B, and stigmasterol.

31. The method of claim 28, wherein said at least one purified compound for each of (i), (ii) and (iii) is independently selected from quercetin, luteolin or kaempferol.

32. The method of claim 28, wherein said at least one purified compound for each of (i), (ii) and (iii) is luteolin.


SEQUENCE LISTING (10 sequences)

SEQ ID NO: 1 - DNA, 27 bp, synthetic construct
  misc_feature 1..27: note = Synthetic Oligonucleotide
  source 1..27: mol_type = other DNA
ctaggactgttaggcaccgagcctggc

SEQ ID NO: 2 - DNA, 33 bp, synthetic construct
  misc_feature 1..33: note = Synthetic Oligonucleotide
  source 1..33: mol_type = other DNA
ccacttttacaggcaatcaagaaaaataactgc

SEQ ID NO: 3 - DNA, 22 bp, synthetic construct
  misc_feature 1..22: note = Synthetic Oligonucleotide
  source 1..22: mol_type = other DNA
ggtgcctatgtctcagcctctt

SEQ ID NO: 4 - DNA, 23 bp, synthetic construct
  misc_feature 1..23: note = Synthetic Oligonucleotide
  source 1..23: mol_type = other DNA
gccatagaactgatgagagggag

SEQ ID NO: 5 - DNA, 22 bp, synthetic construct
  misc_feature 1..22: note = Synthetic Oligonucleotide
  source 1..22: mol_type = other DNA
cctgtggatgactgagtacctg

SEQ ID NO: 6 - DNA, 23 bp, synthetic construct
  misc_feature 1..23: note = Synthetic Oligonucleotide
  source 1..23: mol_type = other DNA
agccaggagaaatcaaacagagg

SEQ ID NO: 7 - DNA, 22 bp, synthetic construct
  misc_feature 1..22: note = Synthetic Oligonucleotide
  source 1..22: mol_type = other DNA
tggaccttccaggatgaggaca

SEQ ID NO: 8 - DNA, 21 bp, synthetic construct
  misc_feature 1..21: note = Synthetic Oligonucleotide
  source 1..21: mol_type = other DNA
gttcatctcggagcctgtgtg

SEQ ID NO: 9 - DNA, 18 bp, synthetic construct
  misc_feature 1..18: note = Synthetic Oligonucleotide
  source 1..18: mol_type = other DNA
tttcaagggtgccagttt

SEQ ID NO: 10 - DNA, 18 bp, synthetic construct
  misc_feature 1..18: note = Synthetic Oligonucleotide
  source 1..18: mol_type = other DNA
gaggccagcatcgtgtag